(19) United States
(12) Patent Application Publication
(10) Pub. No.: US 2010/0217250 A1
(43) Pub. Date: Aug. 26, 2010

(54) METHODS AND SYSTEMS FOR CONTROLLED THERMAL TISSUE

(75) Inventor: Russel M. Sampson, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Sierra Surgical Technologies, Palo Alto, CA (US)

(21) Appl. No.: 12/711,770

(22) Filed: Feb. 24, 2010

Related U.S. Application Data
(60) Provisional application No. 61/154,898, filed on Feb. 24, 2009.

(51) Int. Cl. A61B 18/08

(52) U.S. Cl. 606/29; 606/27

(57) ABSTRACT

A body passage having an interior wall with a lining is occluded by introducing a thermal delivery catheter to the passage. The thermal delivery catheter has a thermal transfer region which can deliver both a coagulative tissue necrosis energy dosage and a thermally fixing energy dosage. The coagulative necrosis dosage will result in scar tissue formation, while the thermally fixing tissue dosage will prevent regrowth of the tissue lining from neighboring untreated tissue regions which could compromise the integrity of the occlusion which is formed.

### CROSS-REFERENCES TO RELATED APPLICATIONS

This application claims the benefit of provisional application No. 61/154,898 (Attorney Docket No. 026916-000300US), filed on Feb. 24, 2009, the full disclosure of which is incorporated herein by reference.

### BACKGROUND OF THE INVENTION

1. Field of the Invention

The present invention relates generally to medical methods and devices. More particularly, the present invention relates to methods and devices for occluding a body passage by inducing coagulative tissue necrosis in combination with thermally fixing a peripheral zone of tissue within the passage.

The application of radiofrequency and other energy sources to tissue for tissue ablation has been utilized for a number of purposes. Of particular interest, the use of radiofrequency energy to treat tissue and block passage of eggs through the Fallopian tube into the uterus has been proposed for achieving contraception. For example, U.S. Pat. No. 7,220,259 describes a plug having a plurality of electrodes on its surface which is introduced into the uterotubal junction at the transition from the Fallopian tube to the uterus. Radiofrequency energy is applied through the electrodes to cause tissue to constrict around the plug and block passage of the egg. U.S. Patent Publ. No. 2006/0135956-A1 describes a radiofrequency device having bipolar electrodes at its distal end. The device is advanced to the cornu which is the region at the corner of a uterus adjacent to the Fallopian tube os. Radiofrequency energy is applied through the electrodes to ablate tissue to a known depth, causing a healing response which causes scarring and occludes the opening from the Fallopian tube to the uterus.

While very promising, both these techniques can fail if the endometrium of the uterus or the endothelial layer of the Fallopian tube regenerate and create passages bypassing the plug or through the scar tissue. In such cases, the sperm could pass through the uterus into the fallopian tube where the egg is present, allowing the sperm to fertilize the egg and pregnancy to occur.

For these reasons, it would be desirable to provide methods and devices for applying energy to the tissue lining body passages in such a way that permanent occlusion of the passages may be reliably obtained with a reduced risk of the tissue lining regenerating to compromise the occlusion. In particular, it would be desirable if the energy could be applied in a manner which both induces scaring and occlusion while inhibiting the regeneration of an endothelial, endometrial, or other tissue lining which can compromise the ability to achieve permanent occlusion. Preferably, these objectives can be met through the use of a single device that would be compatible both with the use of implants and with protocols which do not require the use of an implant. At least some of these objectives will be met by the inventions described hereinbelow.

2. Description of the Background Art

U.S. Pat. No. 7,220,250 and Published U.S. Patent Application No. 2006/013956 have been described above. Other patents and applications of interest include U.S. Pat. No. 6,258,084 and Published U.S. Patent Application Nos. 2009/056722; 2009/054884; and 2008/154256.

### BRIEF SUMMARY OF THE INVENTION

The present invention provides methods and devices for occluding body passages, particularly including the distal cornu of a uterus, the Fallopian tube ostia at the ends of the cornu which open into the Fallopian tubes, and interstitial regions of the Fallopian tubes. The methods rely on both creating an area of coagulative tissue necrosis within the body passage and thermally fixing a peripheral zone of tissue over at least a portion of the wall of the body passage surrounding or adjacent to the coagulative tissue necrosis. The coagulative tissue necrosis will result in the formation of scar tissue which will fully occlude the body passage over time, while the more immediate thermal tissue fixation will inhibit the regrowth of a tissue lining across the area of coagulative tissue necrosis, such as an endothelial layer or an endometrial layer, which can compromise the integrity of the occlusion.

By “coagulative tissue necrosis,” it is meant that the tissue is treated to create a reversible necrosis, typically by exposing the tissue to a thermal insult, usually by the application of radiofrequency energy, or other energy, to the lining surface of the tissue surrounding a target site within the body passage. Such reversible necrosis provides coagulative tissue necrosis by exposing the tissue to a thermal history (heating the tissue to a sufficient temperature for a sufficient duration) to induce necrosis, without causing a transition to thermal fixation, or permanent necrosis. For example, for tissue lining the Fallopian tubes, treatment at a temperature in the range from 55° C. to 70° C. for a time in the range from 10 seconds to 120 seconds will typically be sufficient to induce reversible necrosis leading to coagulative tissue necrosis and occlusive scarring. For tissue surrounding the uterus, typically the endometrial tissue at the distal corner of the uterus, treatment at a temperature in the range from 50 C to 75 C for a time in the range from 10 seconds to 120 seconds will typically be sufficient to cause a deep reversible necrosis which is most likely to achieve the coagulative tissue necrosis which causes the scar formation while avoiding permanent tissue necrosis. The treatment conditions are intended to be exemplary and other conditions might also find use. Coagulative tissue necrosis according to the present invention will preferably be induced over a length of the body passage which is sufficient to assure the desired passage occlusion. Typically, the length will be in the range from 2 mm to 20 mm. Within the Fallopian tube, the length of the occlusion will be in the range from 2 mm to 10 mm, while within the distal cornu of the uterus, the length will typically be in the range from 5 mm to 15 mm.

By “thermal tissue fixation,” it is meant that the tissue is treated, typically by inducing thermal injury, to cause a permanent and immediate necrosis of the tissue lining, such as the endothelial lining or the endometrial lining, under conditions where regrowth of the lining will be prevented. Thermally fixed tissue is considered a foreign body during the tissue healing process. The tissue cannot be broken down and absorbed and/or regenerated as it is with coagulative necrosis. It acts as a barrier or blockade to the advance of a tissue healing response. By placing a zone of thermally fixed tissue between coatulative necrosis tissue and surrounding untreated tissue, the thermally fixed tissue acts as a barrier to prevent the untreated tissue from acting to induce a re-epithelialization of the coagulative necrosis tissue which would inhibit scar formation. Typically, treatment temperatures in the range from 70° C. to 100° C. for a time in the range from 10 seconds to 60 seconds will be utilized. An object of the present invention is to inhibit regrowth of the tissue lining, and the thermal tissue fixation will be induced in a zone that can be limited to a relatively narrow stripe or ring extending peripherally or circumferentially over at least a portion of the wall of the body passage being treated, usually extending contiguously over a complete circumferential path surrounding the passage, separating surrounding untreated tissue from the area of coagulative necrosis. Typically, the stripe or ring will have a width in the range from 1 mm to 5 mm.

In a first aspect of the present invention, a method for occluding a body passage having an interior wall with a lining comprises inducing coagulative tissue necrosis at a location in the passage. A peripheral stripe of tissue is then thermally fixed over at least a portion of the interior wall adjacent to or overlapping with the necrosed location. The healing and scaring response to coagulative tissue necrosis will occlude the passage, but would be subject to the regrowth of the tissue lining in the absence of the thermal tissue fixation. The thermal tissue fixation will prevent regrowth of the tissue lining from neighboring untreated tissue for a time sufficient to allow the coagulative tissue necrosis to result in scar tissue being formed within the body passage to fully occlude said passage.

Typically, the methods of the present invention will be performed by introducing an energy transfer device to a location within the body passage being treated. The energy transfer device will be engaged against the interior wall of the passage proximate the location to the area to be treated and delivering both a coagulative energy dosage and a thermally fixing energy dosage from the energy transfer device. Energy transfer device may comprise a plurality of axially spaced-apart ring electrode structures which may be selectively energized at different times to provide the coagulative energy dosage and the thermally fixing energy doses. Alternatively, the energy transfer device may comprise two or more energy transfer regions adapted to deliver the coagulative energy dosage and the thermally fixing energy dosage simultaneously from different locations on the device.

For treating the cornu of a uterus, the energy transfer device will usually be conformed to the shape of the cornu, generally a triangular shape, so that it closely engages the endometrium lining the cornu prior to delivering the energy dosages. For treating a Fallopian tube, energy transfer device may be more cylindrical in shape, and may be introduced into an interstitial region of the Fallopian tube prior to delivering the energy dosages. After treatment of the Fallopian tube, the energy transfer device may be removed or, alternatively, the energy transfer device may be left as a permanent implant within the interstitial region. As an additional alternative, the energy transfer device may be removed and a separate material may be left as a permanent and/or absorbable implant.

For inducing coagulative tissue necrosis, the energy transfer device will typically deliver radiofrequency energy at a power of 5 to 10 Watts and energy density in the range from 60 J/cm² to 150 J/cm². Thermally fixing the contiguous stripe of tissue may comprise delivering radiofrequency energy from the transfer device at a power of 10 to 30 Watts and energy density in the range from 100 J/cm² to 200 J/cm².

In a further aspect of the present invention, systems for delivering energy to occlude a body passage comprise a catheter adapted to be transcervically introduced to a uterus, an energy transfer surface at a distal end of the catheter, and a power supply connectable to the catheter and programmable to deliver both a thermally fixing energy dosage to the energy transfer surface and a coagulative necrosis energy dosage to the energy transfer surface. The energy transfer surface may comprise a plurality of axially spaced-apart ring electrode structures and the power supply may comprise switching circuitry which may be selectively configured to deliver bipolar radiofrequency energy to pairs of said ring electrode structures to provide both the thermally fixing energy dosage and the coagulative necrosis energy dosage. The switching circuitry may be implemented entirely in hardware with mechanical or solid state switches, but will typically be implemented at least partially in logic or software which controls the physical switches.

In an alternative embodiment, the energy transfer surface of a catheter may comprise an electrode array and an electrically resistive cover over a portion of said array. The power supply will typically be adapted to deliver radiofrequency energy to the electrode array, usually the generally constant power density, and the electrically resistive cover will create a low energy transfer region and a high energy transfer region, where the low energy transfer region delivers the coagulative necrosis energy dosage while the high energy transfer region delivers the thermally fixing energy dosage.

In a still further aspect of the present invention, an energy delivery catheter comprises a catheter body having a proximal end, a distal end, and being adapted to be transcervically introduced to a uterus. An electrode support structure on the distal end of the catheter has a surface which can be expanded to conform to a cornu of the uterus. An electrode array on the surface of the support structure will usually comprise at least four axially spaced-apart ring electrode structures which are expandable to engage endometrial tissue of the cornu when the support is expanded. The catheter will usually include at least four electrically isolated electrical conductors with at least one such conductor connected to each of the four ring electrode structures to allow the electrodes to be selectively energized in a variety of patterns to effect the desired low power transfer and high power transfer. Preferably, the catheter body will be curved so that it will conform to a side of the uterus from the cervical os to the cornu, and the electrode support will preferably radially expand outwardly relative to the curve of the catheter body. The electrode support may be configured to expand to a triangular profile with a peak directed radially outwardly relative to the curve of the catheter body so that the electrode supported on the expanded support will conform to the generally triangular cornu. The electrode support may have a variety of configurations, typically being a mechanically expansible cage or other structure, such as a deflectable member comprised of nitinol or stainless steel, or alternatively an inflatable member.

In yet another aspect of the present invention, an energy delivery catheter comprises a catheter body having a proximal end, a distal end, and being adapted to being transcervically introduced into the uterus. An electrode structure at the distal end of the catheter body includes an electrode array and an electrically resistive cover over a portion of the array. The cover creates a low energy transfer region to deliver a coagulative tissue necrosis dosage and an axially offset high energy transfer region to deliver a thermally fixing energy dosage. In this way, energy can be simultaneously delivered to the target tissue to provide both the coagulative necrosis and thermal fixation at the same time. The catheter body may be formed generally as the prior embodiment having a curve which conforms to a side of the uterus from the cervical os to the cornu. The electrically resistive cover may have a variety of configurations, typically being made from materials such as a composite of nylon and polyurethane. Usually, the electrode array will comprise at least two axially oriented bipolar electrode pairs which may be independently connected to a power supply using independent conductors.

### BRIEF DESCRIPTION OF THE DRAWINGS

FIG. 1 illustrates a system of the present invention including a thermal delivery catheter having an energy transfer surface at its distal end connected to a power supply.

FIGS. 2A and 2B illustrate a first embodiment of an energy transfer surface that can be utilized in the thermal delivery catheter of the system of FIG. 1.

FIG. 3 illustrates a second embodiment of the energy transfer surface that can be utilized in the thermal delivery catheter of the system of FIG. 1.

FIGS. 4, 4A and 4B illustrate a third embodiment of the energy transfer surface that can be utilized in the energy transfer catheter of the system of FIG. 1.

FIGS. 5A-5D illustrate use of the thermal delivery catheter of FIGS. 1, 2A and 2B for treating and occluding a distal cornu of a uterus in accordance with the principles of the present invention.

FIG. 6 illustrates a switching circuit that can be employed in the power supply of the system of FIG. 1 for delivering energy using the catheters shown in FIGS. 5A-5D.

FIGS. 7A and 7B illustrate two different electrode energization schemes that can be utilized and implemented with the switching circuitry of FIG. 6 for treatment as shown in FIGS. 5A-5B.

FIGS. 8A-8D illustrate the progressive treatment of tissue utilizing the electrode energization pattern of FIG. 7A.

FIGS. 9A-9D illustrate the progressive tissue treatment utilizing the electrode energization pattern of FIG. 7B.

FIGS. 10 and 11A-11C illustrate treatment of an interstitial region of a Fallopian tube using the energy transfer catheter of FIG. 3.

FIGS. 12A-12D illustrate treatment of a distal cornu of a uterus using the thermal treatment catheter of FIGS. 4, 4A and 4B.

### DETAILED DESCRIPTION OF THE INVENTION

Providing permanent necrosis in a body passage while creating a barrier to endothelial or endometrial regrowth is achieved by the selective application of energy, typically thermal energy. Any body lumen having an endothelial or endometrial lining can be treated. A single device can be inserted that treats multiple regions of the lumen or body passage to achieve different effects. For example, the catheter or other device may be inserted at least partially into the body passage or lumen, where portions of the device are controlled or managed separately to deliver different energy dosages, typically by delivering different power densities and/or similar power densities over different time periods to create different thermal histories in different regions of the tissue. In at least a portion of the tissue, a thermal history will be induced to create coagulative tissue necrosis with the formation of scar tissue to occlude the body lumen or passage. In other regions, the tissue will be subjected to a thermal history to induce thermal fixation in order to form a barrier between untreated tissue and coagulative necrosis tissue, which will prevent the regrowth of the endothelial or endometrial lining, which regrowth can compromise the ability of the coagulative necrosis to completely occlude the lumen.

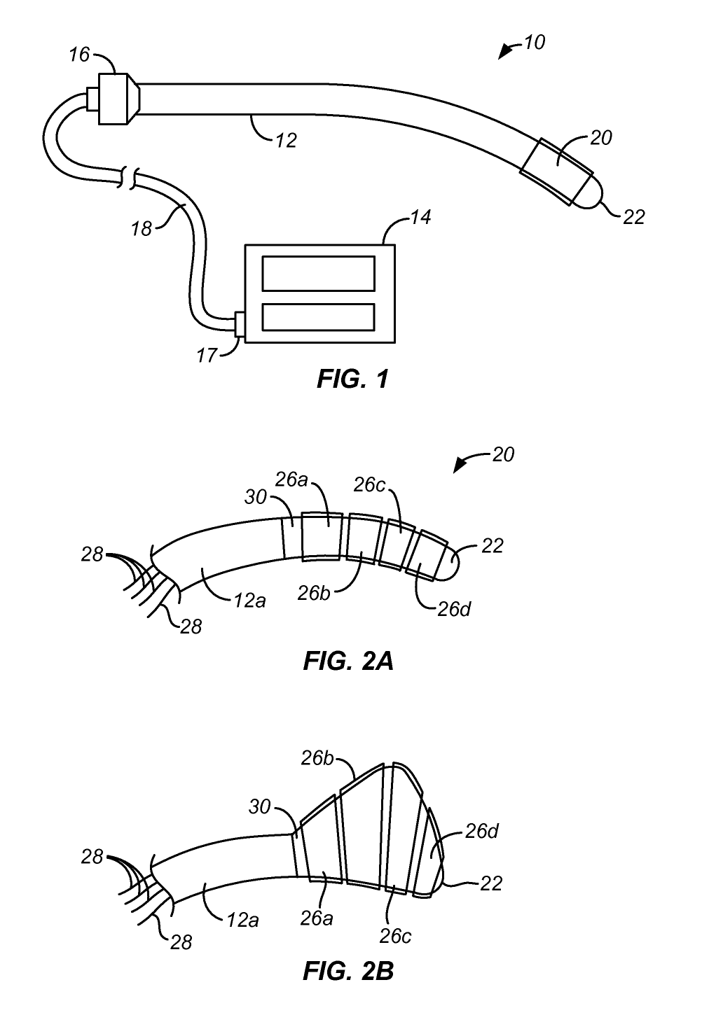

An exemplary system for treating tissue in accordance with the principles of the present invention is illustrated in FIG. 1. A system 10 comprises a thermal delivery catheter 12 and a power supply 14. The catheter 12 is connected to the power supply by a cable 18 and proximal connector 16. The power supply 14 will typically generate radiofrequency energy, but other forms of energy including microwave, direct current for resistive heating, ultrasound, optical (laser) energy, and the like, could also be used with proper modification of other components of the system.

A thermal delivery catheter 12 includes an energy transfer surface 20 at or near its distal end 22. The energy transfer surface will be adapted to deliver energy from the power supply 14 into a tissue surface against which the energy delivery surface 20 is engaged. The energy transfer surface 20 may have a wide variety of configurations which depend, at least in part, on the type of energy being delivered. For the delivery of radiofrequency energy, the energy transfer surface 20 will typically comprise at least two electrodes to deliver bipolar energy into the tissue. Although it will be possible to employ monopolar energy using only a single electrode, the delivery of bipolar radiofrequency energy is generally preferred as it can be more carefully confined to the target tissue and the power density can be more readily controlled.

Referring now to FIGS. 2A and 2B, a first exemplary energy transfer surface comprises a plurality of electrode rings 26a-26d, each of which are connected to the proximal hub 16 by an individual conductor 28. The ring electrodes 26a-26d fully circumscribe the body of the thermal delivery catheter 12a and are mounted directly over an expandable support structure 30 which forms the distal end of the catheter. The electrode rings 26a-26d are usually radially expandable so that when the support structure 30 is expanded, as shown in FIG. 2B, the electrodes are able to conform to the surface of the expanded support structure. The support structure 30 may have a variety of expansion mechanisms, typically being a deflectable metallic member. Alternatively, an inflatable member, mechanical cage or other internal scaffold could be provided. The ring electrodes may be formed from a conductive knit mesh or other electrically conductive material that allows for circumferential expansion as the underlying support is expanded. Alternatively, the electric rings could be formed from a wire mesh or metal foil that is loosely attached to the support to allow expansion. A preferred geometry for the support structure 30 is shown in FIG. 2B, having a triangular profile extending in a direction which is radially outward from the curve of the body of the catheter 12a as seen in FIG. 1. The catheter 12a having the energy transfer surface of FIGS. 2A and 2B is intended particularly for treating a distal cornu of the uterus, as described in more detail below.

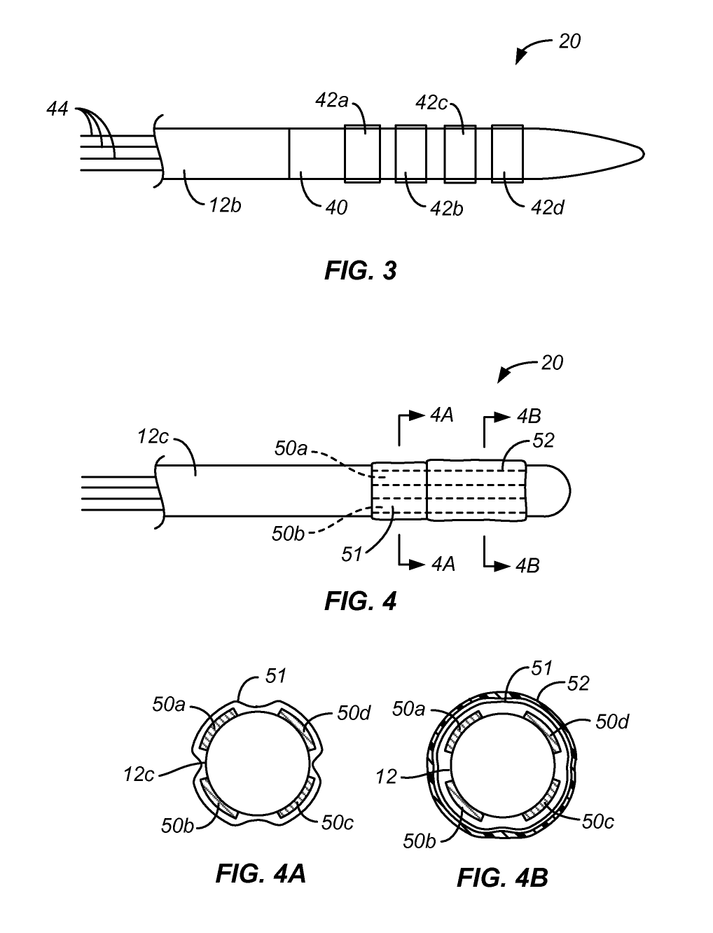

A second embodiment of an energy transfer surface 20 intended for treating an interstitial region of a Fallopian tube is illustrated in FIG. 3. The catheter 12b terminates in a non-expandable support structure 40 which bears four axially spaced-apart electrodes 42a-42d. The catheter 12b of FIG. 3 will generally be dimensioned to be advanced through a Fallopian tube os and into the interstitial region of the Fallopian tube, typically having a diameter in the range from 0.5 mm to 1.5 mm and a length in the range from 5 mm to 20 mm. Optionally, the non-expandable support 40 may be configured to be detachable from the remainder of the catheter 12b so that it can be left in place within the interstitial region of the Fallopian tube after the tissue has been thermally treated, as described in more detail below. As with the prior catheter embodiment, each of the individual electrodes 42a-42d is connected to a conductor 44 which allows connection to the power supply.

A still further embodiment of the thermal delivery catheter of the present invention is illustrated in FIGS. 4, 4A, and 4B. There, the catheter 12c has an energy transfer surface 20 comprising four axially oriented circumferentially spaced-apart electrodes 50a-50d, typically surrounded by a conductive mesh or other array 51 (suitable conductive mesh arras are described, for example, in US2006/0135956, the full disclosure of which is incorporated herein by reference). An electrically resistive cover 52 extends over a distal portion of the axial length of the electrodes 50a-50d, as best seen in FIG. 4. Cover 52 creates a low power transfer region where the cover is present and a high power transfer region where the cover is not disposed over the electrodes 50a-50d. In this way, whenever the electrodes are powered, a distal region will be delivering less energy than a proximal region. By properly choosing the energy density delivered by the electrodes, the distal region will be able to deliver a coagulative necrosis dosage while the proximal region will be able to deliver a thermally fixing dosage, as generally described above. The catheter of FIGS. 4, 4A and 4B can be utilized for treating either a cornu, an interstitial region of the Fallopian tube, or other body passages in accordance with the principles of the present invention.

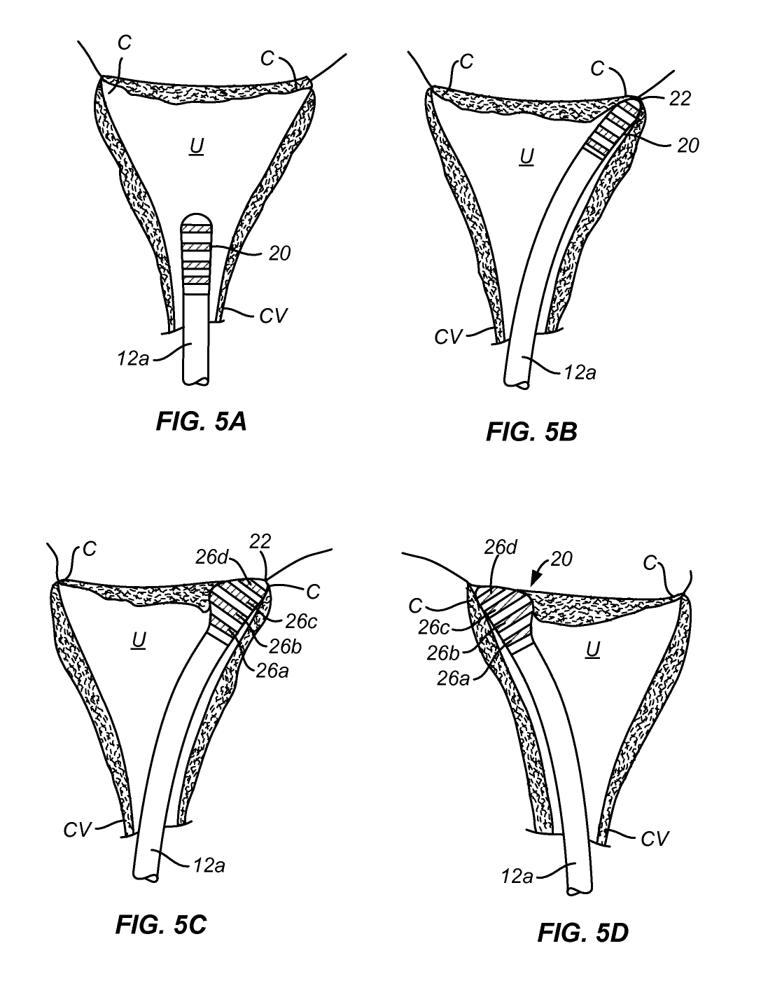

Referring now to FIGS. 5A-5D, the use of the catheter 12a for treating a distal cornu C of a uterus U will be described. Catheter 12a is initially introduced transcervically through the cervix CV, as shown in FIG. 5A. The energy transfer surface 20 is then advanced to a first cornu C as shown in FIG. 5B. The curved length of the catheter body will generally conform to the side of the uterus to help position the distal end 22 of the catheter adjacent the Fallopian tube os. The catheter support 30 is then expanded, as shown in FIG. 5C, and the electrodes 26a-26d then energize to treat the tissue, as will be described in more detail below. After treating the first cornu, the catheter 12a may be rotated to reposition the energy transfer surface 20 at the second cornu, as illustrated in FIG. 5D. The energy transfer surface may then be utilized to treat the second cornu in the same manner as the first cornu. After the treatment has been completed, the catheter support 30 can be contracted and the catheter 12 may be removed from the cervix with occlusion of the cornu following over time.

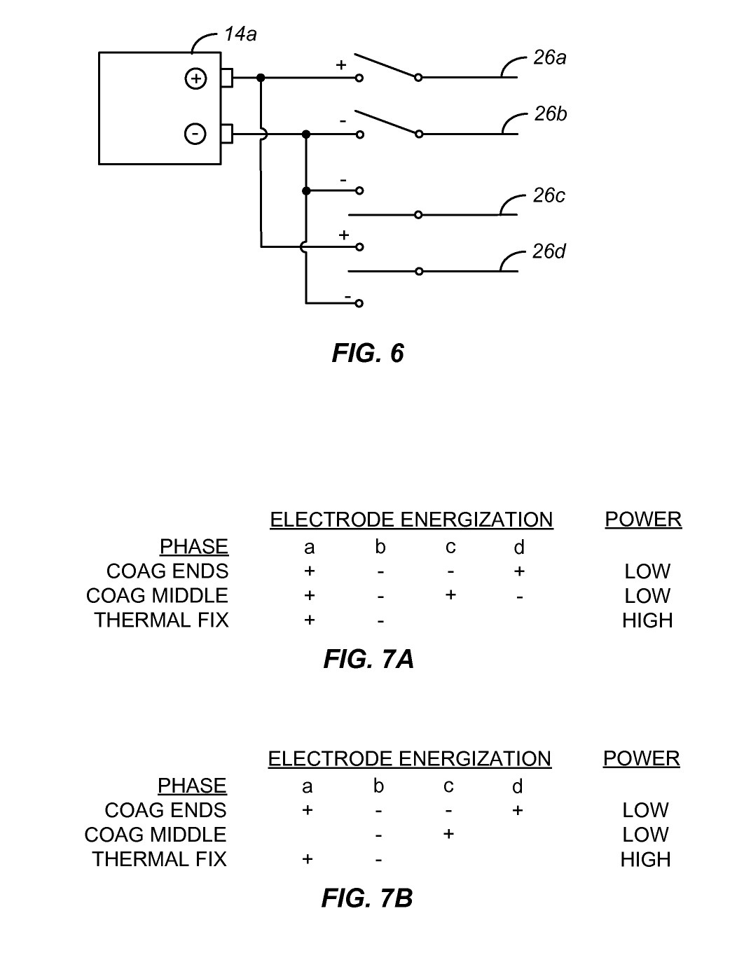

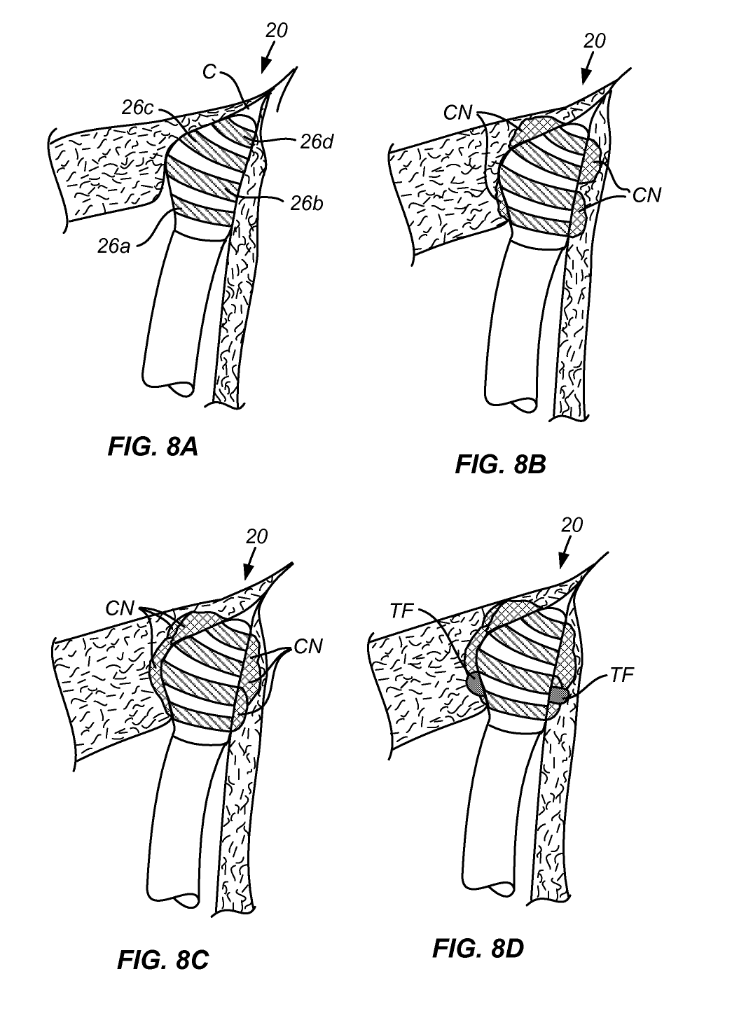

The desired ablation pattern and thermal treatment history of the tissue is achieved by selectively energizing the electrodes 26a-26d, as will be described with reference to FIGS. 6, 7A, and 8A-8D. Initially, the energy transfer surface is positioned at the cornu with the electrodes 26a-26d in a deenergized position, as shown in FIG. 8A. The electrodes are then selectively energized in phases using a switching circuit, as shown in FIG. 6, to achieve an energization pattern, as shown in FIG. 7A. Initially, the individual switches of the switching circuitry of FIG. 6 are closed to deliver energy from the RF power supply 14a, with electrode 26a connected to a positive terminal, electrodes 26b and 26c being connected to negative electrodes, and electrode 26d being connected to a positive electrode to deliver a low energy density in order to coagulate the ends of the tissue to create coagulatively necrosed CN regions, as generally shown in FIG. 8B. Next, electrodes 26a and 26c are connected to a positive terminal while electrodes 26b and 26d are connected to the negative terminal to fill and necrosis in the middle tissue region, as shown in FIG. 8C. At this point, the tissue has been exposed to reversible necrosis and, over time, the scar tissue would form and a coagulative necrosis response would be completed. While in most cases, such treatment would result in complete occlusion, in certain instances, the endometrial lining from the interior of the uterus could regrow and reestablish a path through the scar tissue before complete occlusion has been achieved. In those instances, it would be possible for sperm to travel from the uterus into the Fallopian tube and fertilize an egg residing within the Fallopian tube resulting in pregnancy. In order to prevent such failure, the present invention provides for thermal fixation of a stripe or other zone of tissue TF over at least a portion of the cornu, as illustrated in FIG. 8D. Thermal fixation may be achieved by energizing electrodes 26a and 26b at a high power level in order to irreversibly necrose the tissue and prevent any regeneration of the endometrial layer.

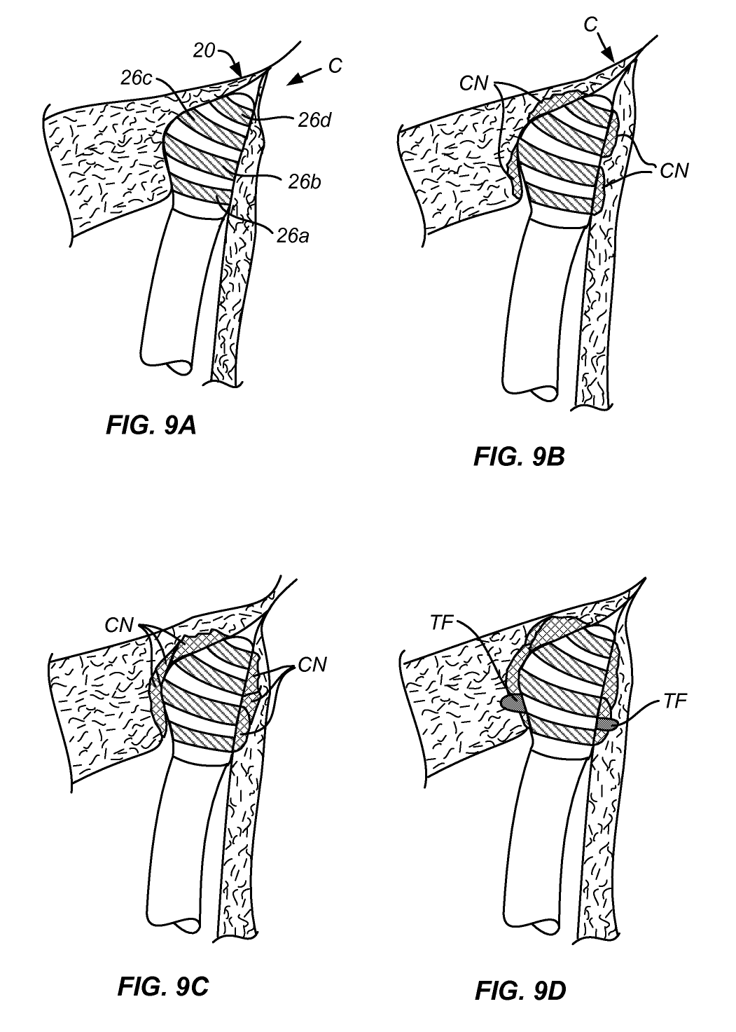

As a slight variation in the protocol illustrated in FIG. 7A, the middle coagulation step may be achieved by energizing only electrodes 26b and 26c, focusing the further coagulation to the region between the initial two coagulation areas. The resulting necrosis patterns are illustrated in FIGS. 9A-9D. It should be understood that there are multiple similar protocols for coagulation steps that are capable of producing the desired coagulative necrosis surrounded by thermally fixed tissue, and the present invention in no way is intended to be limited to the two protocols described herein.

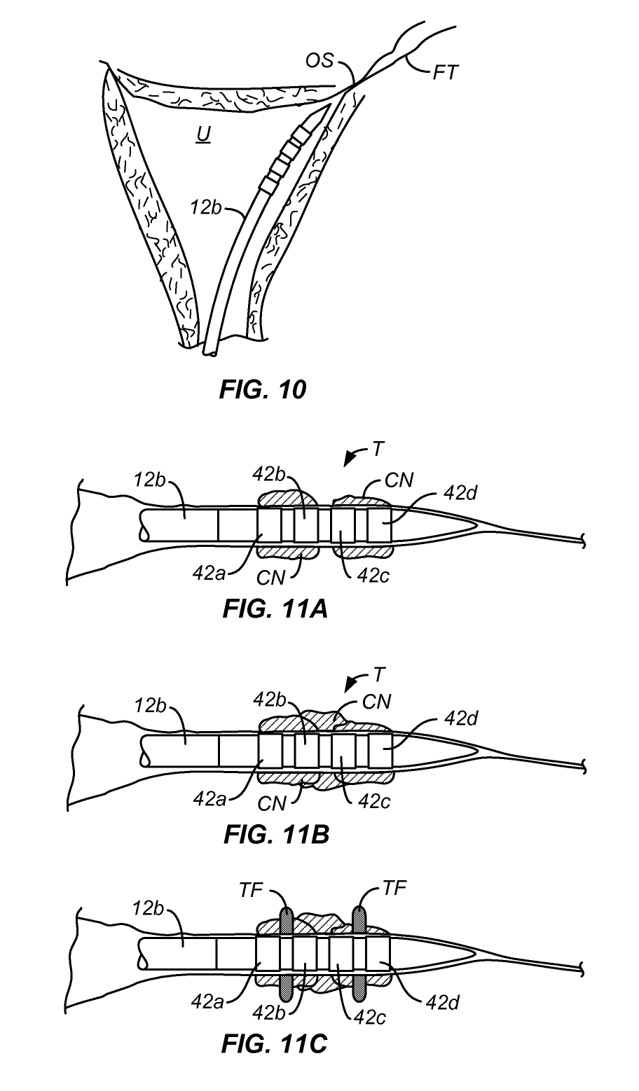

Referring now to FIG. 10, thermal delivery catheter 12b may be introduced into a Fallopian tube by advancing a distal end of the catheter through the Fallopian tube os and into an interstitial region of the tube, as shown in FIG. 11A. The catheter may then be energized according to the pattern set forth in FIG. 7A to first coagulate the ends, as shown in FIG. 11A. Following coagulation of the ends, at least the middle electrodes 42b and 42c are energized in order to close a middle region. Finally, the proximal electrodes 42c and 42d and distal electrodes 42a and 42b are energized at high power to create two thermally fixed tissue barriers TF, as shown in FIG. 11C. By forming the tissue fixation barriers, endothelial tissue within the Fallopian tube from areas adjacent to the treatment zone cannot regrow into the treated zone and disturb the occlusion, allowing the coagulative necrosis tissue to form an occlusion resulting from the ultimate formation of scar tissue. Optionally, only a single thermally fixed barrier could be created by energizing only electrodes 42a and 42b if prevention of regrowth of endothelial tissue from the other side of the Fallopian tube is not needed.

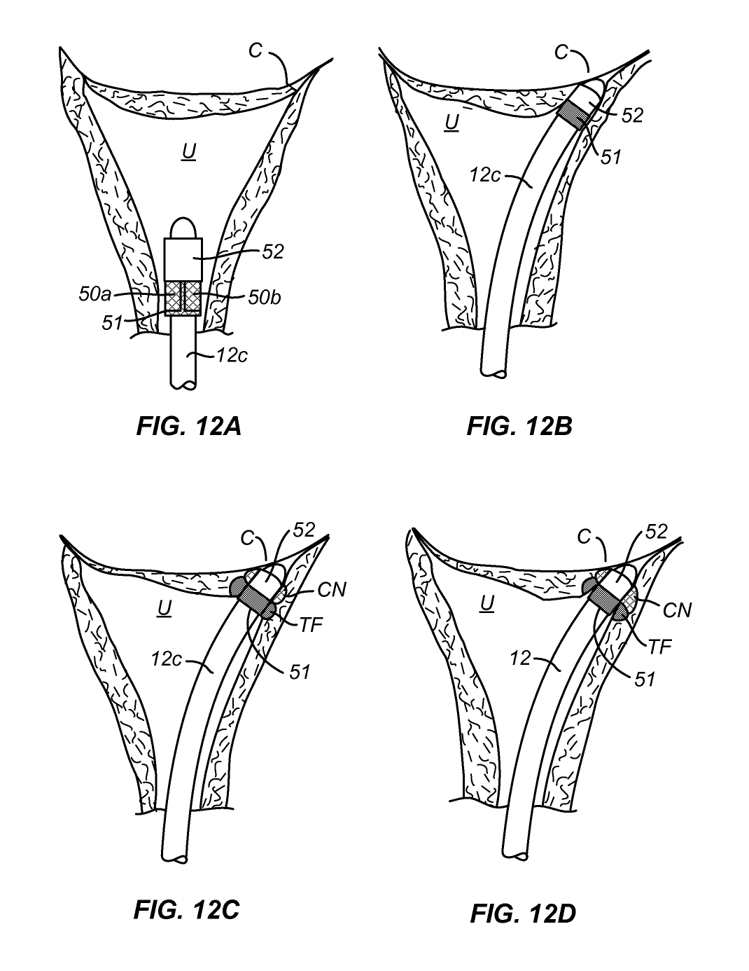

Referring now to FIGS. 12A-12D, use of the thermal delivery catheter 12c, as illustrated in FIGS. 4, 4A, and 4B, for occluding a cornu C of a uterus U will be described. Catheter 12c is transcervically introduced into the uterus, as shown in FIG. 12A, and advanced so that the electrodes 50a-50d are introduced into the distal cornu, as shown in FIG. 12B. Bipolar radiofrequency energy is then delivered into the electrodes 50a through 50d with the energy density being greater where the electrodes are exposed than where they are covered by cover 52. Thus, the tissue will be necrosed with a coagulative necrosis region CN being created simultaneously with a thermally fixed region TF, as shown in FIG. 12C. As time progresses, the coagulative necrosis region CN will grow more slowly than the thermally fixed region TF, as shown in FIG. 12D. Once treatment is ceased, the coagulativly necrosed region CN will begin forming scar tissue while the thermally fixed region TF will prevent the regrowth of endometrial lining which could prevent full occlusion.

While the above is a complete description of the preferred embodiments of the invention, various alternatives, modifications, and equivalents may be used. Therefore, the above description should not be taken as limiting the scope of the invention which is defined by the appended claims

What is claimed is:

1. A method for occluding a body passage having an interior wall with a lining, said method comprising:
inducing coagulative tissue necrosis at a location in said passage; and
thermally fixing a peripheral zone of tissue over at least a portion of an interior wall surrounding or adjacent to the coagulative tissue necrosis;
wherein the coagulative tissue necrosis occludes said passage and the thermally fixed contiguous ring inhibits regrowth of the tissue lining along the interior wall.

2. A method as in claim 1, wherein inducing coagulative tissue necrosis and thermally fixing a contiguous zone of tissue comprise engaging an energy transfer device against the interior wall of the passage proximate the location and delivering both a coagulative energy dosage and a thermally fixing energy dosage from the energy transfer device.

3. A method as in claim 2, wherein the energy transfer device comprises a plurality of axially spaced-apart ring electrode structures which are selectively energized at different times to provide the coagulative energy dosage and the thermally fixing energy dosage.

4. A method as in claim 2, wherein the energy transfer device comprises different energy transfer regions adapted to deliver the coagulative energy dosage and the thermally fixing energy dosage simultaneously.

5. A method as in claim 2, wherein the body passage is at the cornu of a uterus and the lining comprises an endometrium, further comprising conforming the energy transfer device to the shape of the cornu prior to delivering the energy dosages.

6. A method as in claim 2, wherein the body passage is a Fallopian tube and the lining comprises an endothelium, further comprising advancing the energy transfer device into an interstitial region of the Fallopian tube prior to delivering the energy dosages.

7. A method as in claim 6, further comprising removing the energy transfer device after the energy dosages have been delivered and delivering an implant.

8. A method as in claim 6, further comprising leaving the energy transfer device as a permanent implant within the interstitial region.

9. A method as in claim 1, wherein inducing coagulative tissue necrosis comprises delivering radiofrequency energy at a power of 5 to 10 Watts and energy density in the range from 50 J/cm² to 150 J/cm² thermally fixing the contiguous stripe of tissue comprises delivering radiofrequency energy at a power of 15 to 30 Watts and energy density in the range from 100 J/cm² to 200 J/cm².

10. A method as in claim 9, wherein the passage is the cornu of a uterus and the coagulative tissue necrosis is induced over a length of endometrium in the range from 5 mm to 15 mm and the thermally fixed stripe has a width in the range from 1 mm to 5 mm.

11. A method as in claim 9, wherein the passage is an interstitial region of a Fallopian tube and the coagulative tissue necrosis is induced over a length of endothelium in the range from 2 mm to 10 mm and the thermally fixed stripe has a width in the range from 1 mm to 5 mm.

12. A system for delivering energy to occlude a body passage, said system comprising:
a catheter adapted to be transcervically introduced to a uterus;
an energy transfer surface at a distal end of the catheter; and
a power supply connectable to the catheter and programmable to deliver both a thermally fixing energy dosage to the energy transfer surface and a coagulative necrosis energy dosage to the energy transfer surface.

13. A system as in claim 12, wherein the energy transfer surface comprises a plurality of axially spaced-apart ring electrode structures and the power supply comprises switching circuitry which may be selectively configured to deliver bipolar radiofrequency energy to pairs of said ring electrode structures to provide both the thermally fixing energy dosage and the coagulative necrosis energy dosage.

14. A system as in claim 13, wherein the switching circuitry is at least partially implemented by software.

15. A system as in claim 12, wherein the energy transfer surface comprises an electrode array and an electrically resistive cover over a portion thereof wherein the power supply delivers radiofrequency energy to the electrode array and the electrically resistive cover creates a low energy transfer region which delivers the coagulative necrosis energy dosage and a high energy transfer region which delivers the thermally fixing energy dosage.

16. An energy delivery catheter comprising:
a catheter body having a proximal end, a distal end, and adapted to be transcervically introduced into the uterus;
an electrode support structure on the distal end of the catheter body and having a surface which can be expanded to conform to a cornu in the uterus.;
an electrode array on the surface of the support structure, wherein said array comprises at least four axially spaced-apart ring electrode structures which are expandable to engage endometrial tissue of the cornu when the support structure is expanded, and
at least four electrically isolated electrical conductors with at least one such conductor connected to each of the at least four ring electrode structures.

17. A catheter as in claim 16, wherein the catheter body is curved so that it will conform to a side of the uterus from the cervical os to the cornu.

18. A catheter as in claim 17, wherein the electrode support expands radially outwardly relative to the curve of the catheter body.

19. A catheter as in claim 18, wherein the electrode support expands to a triangular profile with a peak directed radially outwardly.

20. A catheter as in claim 16, wherein the electrode support structure is mechanically expansible.

21. An energy delivery catheter comprising:
a catheter body having a proximal end, a distal end, and adapted to be transcervically introduced into the uterus; and
an electrode structure at the distal end of the catheter body, said electrode structure including an electrode array and an electrically resistive cover over a portion of the electrode array to create a low energy transfer region to deliver a coagulative tissue necrosis dosage and an axially offset high energy transfer region to deliver a thermally fixing energy dosage.

22. A catheter as in claim 21, wherein the catheter body is curved so that it will conform to a side of the uterus from the cervical os to the cornu.

23. A catheter as in claim 21, wherein the electrically resistive cover comprises a composite of nylon and polyurethane.

24. A catheter as in claim 21, wherein the electrode array comprises at least two axially oriented bipolar electrode pairs.
